(19) United States
(12) Patent Application Publication
(10) Pub. No.: US 2007/0134786 A1
(43) Pub. Date: Jun. 14, 2007

(54) Assaying Device for Determining Activity Value of Nattokinase Solution

(76) Inventors: Wen-Jen Yang, Bade City (TW); Chia-Wei Chang, Bade City (TW); Chih-Yi Lu, Jhongpu Township (TW); Jiunn-Ming Jeng, Sinjhuang City (TW)

Correspondence Address: HDSL, 4331 STEVENS BATTLE LANE, FAIRFAX, VA, 22033, US

(21) Appl. No.: 11/608,832

(22) Filed: Dec. 10, 2006

(30) Foreign Application Priority Data
Dec. 13, 2005 (TW) 094143952

(51) Int. Cl. C12M 1/34

(52) U.S. Cl. 435/288.1; 435/288.3; 435/288.5

(57) ABSTRACT

An assaying device for determining the activity value of Nattokinase solution includes a container having a testing solution therein. Because the testing solution can react with a Nattokinase solution and the reacted testing solution has a different color from the original testing solution, when the testing solution contacts a Nattokinase solution, the reaction occurs along the container. By measuring the length of the reacted testing solution, the activity value of the Nattokinase solution can be easily and quickly obtained.

### BACKGROUND

The invention relates to an assaying device for determining the activity value of Nattokinase solution, and in particular to an assaying device which can quickly and efficiently determine the activity value of Nattokinase solution.

Recently Nattokinase has been proved that it can prevent people from generating thrombus so as to reduce the possibility of cerebral hemorrhage. Therefore, many institutes and companies would like to get into the market of making mass production of Nattokinase. Accordingly, how to identify the quality of Nattokinase is a crucial challenge. Researchers have found that the activity value of Nattokinase solution is a reliable index to determine the quality of Nattokinase.

So called activity value of Nattokinase solution means the concentration of Nattokinase in per volume of Nattokinase sample (solution), which is a concept of relative value but not absolute value. For unity consideration, Japan Nattokinase Association established a standard method for the market to determine the standard activity value of Nattokinase solution.

Thus, if one wants to know the standard activity value of new Nattokinase product, the sample of the product has to be submitted to Japan Nattokinase Association to test and obtain the standard activity value. However, this way not only wastes time but also costs a lot of money. Especially if a Nattokinase product is under development and continuously needs to be improved according to the assayed activity value, this way is not expedience. Accordingly there is a conventional assaying method widely applied, as shown in FIG. 1A to FIG. 1F.

Referring to FIG. 1A first, a culture dish 10 and a beaker 12 containing a testing solution 11 are prepared. The testing solution 11, such as Fibrin solution brought up by Japan Nattokinase Association, is of liquid phase and would react with Nattokinase. Referring to FIG. 1B, the testing solution 11 in the beaker 12 then is poured into the culture dish 10, and is not spilled over the culture dish 10. Next, the surface of the testing solution 11 is flattened as shown in FIG. 1C. The flattened surface can be attained by surface tension thereof resulting from being placed statically for a time period, or attained by slightly vibration. Simultaneously, the testing solution 11 gradually becomes gluey. Next, via a dropper 13, an appropriate amount of Nattokinase solution 14 is dropped in the testing solution 11 as shown in FIG. 1D. Referring to FIG. 1E, because the testing solution 11 reacts with the Nattokinase and changes its color accordingly, the reacted portion of the testing solution 11 becomes a reacted testing solution 11′. Also with reference to FIG. 1F, with visual observation of the difference between the color of testing solution 11 and that of the reacted testing solution 11′, one can easily measure the scope of the reacted solution 11′. If a standard Nattokinase solution with a given standard activity value implements this method, one can further determine and obtain the standard activity value of the Nattokinase solution 14 by comparing with the standard Nattokinase solution.

Although this method can preliminarily obtain the activity value of a Nattokinase solution, there are still a lot of difficulties and disadvantages during carrying out this method. For example, due to the sticky property of the testing solution 11, it is difficult to control the amount of testing solution 11 pouring into the culture dish 10. If all of the Nattokinase samples expected to be identified have different basic experimental conditions, they will not have reliable activity values from comparison procedure. Not mention to that the testing solution 11 becomes sticker as glue. Besides, because the testing solution 11 is quite sticky, the thickness of the testing solution 11 poured in the culture dish 10 will not be uniform, which further complicates experimental conditions.

### BRIEF SUMMARY

The present invention is to provide an assaying device which can quickly determine the activity value of Nattokinase solution, and effectively control the variances of the basic experimental conditions so as to prevent measuring error.

According to the above, the assaying device of the present invention includes a container having a first opening and a receiving room, and a testing solution filled in the receiving room. When the first opening having the testing solution contacts a Nattokinase solution, a reaction occurs in the container.

According to the above, the container is in a long pattern, for example, a tubular container. Thus, the container has the same cross-section in one axis extending from the first opening toward the opposite end of the container. Furthermore, the container has calibrations thereon.

According to the above, the container further comprises a first substrate, a second substrate disposed in parallel with the first substrate to form the receiving room therebetween, and a plurality of spacers disposed between the first substrate and the second substrate, wherein each of the spacers contacts the first substrate and the second substrate, the scale of spacer is not larger than 2 mm, and at least one of the first substrate and the second substrate is a transparent glass.

According to the above, the container further comprises an expanding entrance connected with the first opening.

According to the above, the color of the reacted testing solution is different from that of the original testing solution.

According to the above, the testing solution is filled into the receiving room from the first opening by means of vacuum filling.

According to the above, the container further comprises a second opening located opposite the first opening, and the testing solution is filled into the receiving room from the first opening.

According to the above, two ends of the container is cut off.

### BRIEF DESCRIPTION OF THE DRAWINGS

These and other features and advantages of the various embodiments disclosed herein will be better understood with respect to the following description and drawings, in which like numbers refer to like parts throughout, and in which:

FIGS. 1A to 1F are schematic views of conventional implement steps of determining the activity value of Nattokinase;

FIG. 2A is an exploded view of the assaying device of the first embodiment according to the present invention;

FIG. 2B is a cross-sectional view taken along A-A′ line from FIG. 2A;

FIG. 2C is a schematic view of the assaying device of the first embodiment according to the present invention which has calibrations thereon;

FIG. 2D is schematic view showing the assaying device of the first embodiment used to determine the activity value of a Nattokinase solution;

FIG. 3A is a schematic view of the assaying device of the second embodiment according to the present invention without a testing solution filled therein;

FIG. 3B shows the assaying device of FIG. 3A having a testing solution therein;

FIG. 3C is a schematic view showing the assaying device of the second embodiment used to determine the activity value of a Nattokinase solution; and

FIG. 4 shows the assaying device of the second embodiment further having an expanding entrance.

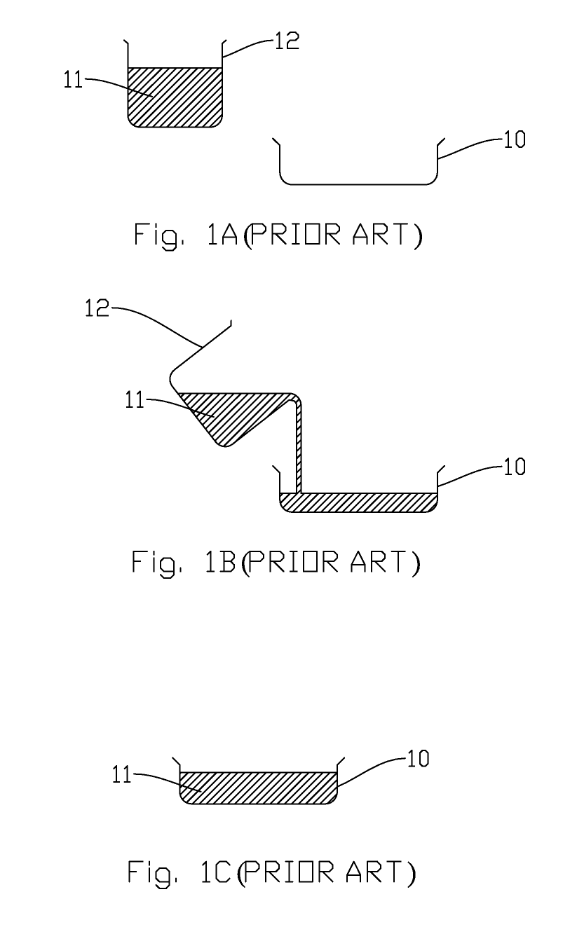

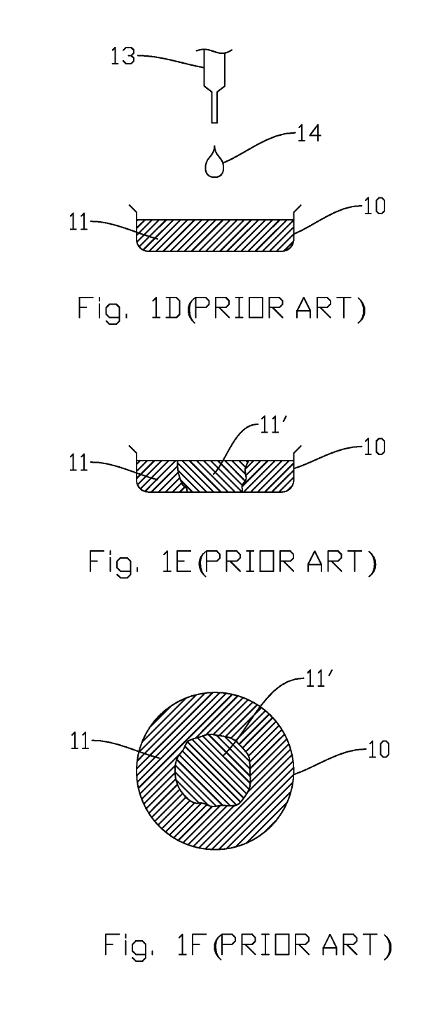

### DETAILED DESCRIPTION

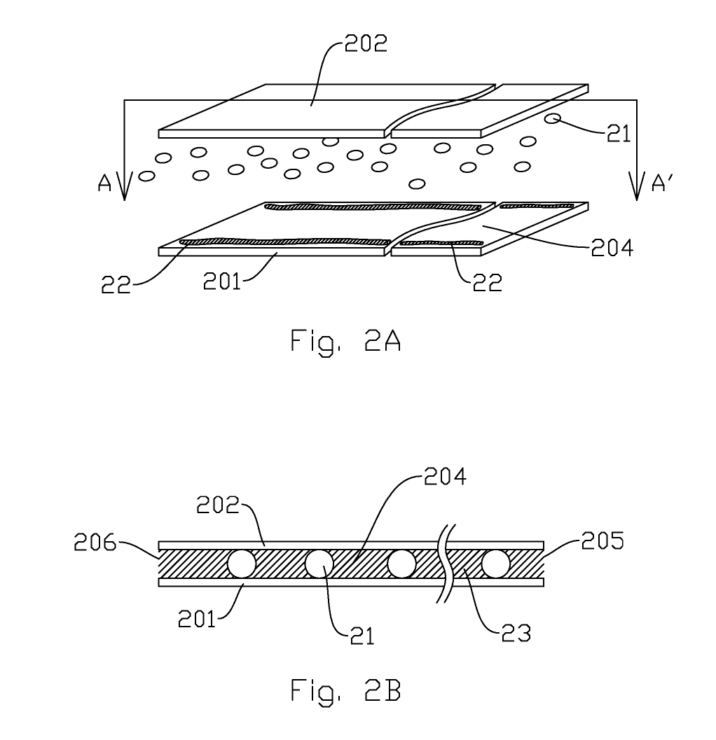

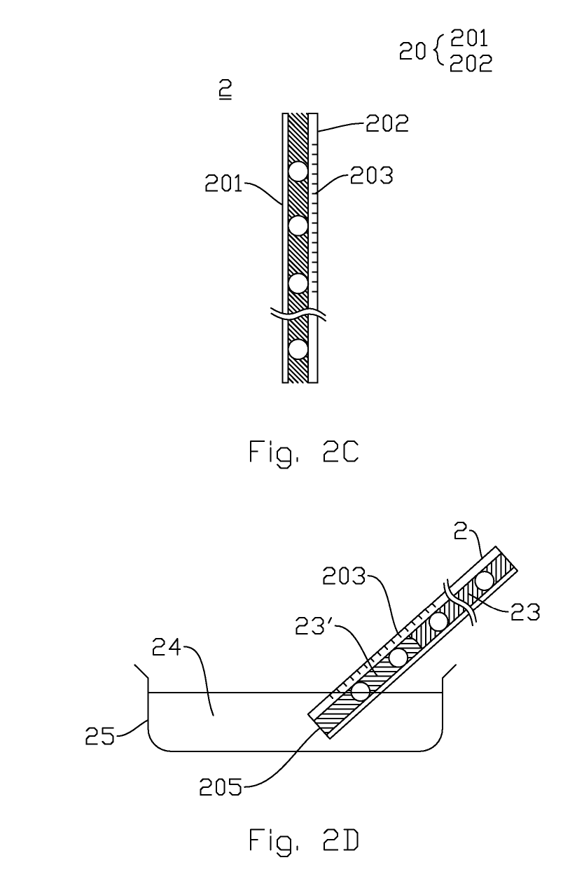

The assaying device for determining the activity value of Nattokinase solution of the first embodiment of the present invention is shown in FIGS. 2A to 2D. Referring to 2A, the assaying device 2 of the first embodiment includes a first substrate 201, a second substrate 202 and a plurality of spacers 21. The first substrate 201 and the second substrate 202 are disposed in parallel, and the spacers 21 are evenly distributed between the first substrate 201 and the second substrate 202 as supports to form a receiving room 204 therebetween. Then a testing solution 23 is filled into the receiving room 204, as shown in FIG. 2B. At least one of the first substrate 201 and the second substrate 202 is made of transparent material; preferably transparent glass. Each of the spacers 21 contacts the first substrate 201 and the second substrate 202, and its scale is controlled not larger than 2 mm, so the breadth of the receiving room 204 is not larger than 2 mm to ensure measuring accuracy. Preferably, an edge adhesive 22 is further disposed between the first substrate 201 and the second substrate 202 to define the receiving room 204, and to form a first opening 205 and a second opening 206, respectively, at opposite sides of the assaying device 2. Then the testing solution 23 can be filled into the receiving room 204 from the first opening 205. The assaying device 2 is preferably used after two ends of both of the first substrate 201 and the second substrate 202 are cut off, as shown in FIG. 2B. In another way, the edge adhesive 22 is to define the receiving room 204 and to form only a first opening 205 (not shown in figures), and then the testing solution 23 is filled into the receiving room 204 by vacuum filling from the first opening 205; that is, the air in the receiving room 204 is draw out to be vacuum first, and then the assaying device 2 is placed in a receptacle having the testing solution 23 to make the testing solution 23 be suck in the receiving room 204 due to the effect of pressure difference. No matter which way is used, the testing solution 23 may be a prior art as mentioned above, which first is of liquid phase and gradually becomes gluey phase during a certain time and will not spill over the receiving room 204. The testing solution 23 can react with Nattokinase and the reacted testing solution has different color from that of the original testing solution.

FIG. 2D is schematic view showing the assaying device 2 applied to a Nattokinase solution 24 with unknown activity value which is placed in a tank 24. The first opening 205 of the assaying device 2 is directly inserted in the Nattokinase solution 24. When the testing solution 23 in the first opening 205 contacts and reacts with the Nattokinase solution 24, a portion of the testing solution 23 becomes a reacted testing solution 23′ extending from the first opening 205 toward the inside of the assaying device 2. Since the reacted testing solution 23′ and the testing solution 23 have different colors, and at least one of the first substrate 201 and the second substrate 202 is transparent, one can easily observe and measure the length of the reacted testing solution 23′ through the first substrate 201 or the second substrate 202. If some Nattokinase solutions with standard activity values are also taken to run the process of the present invention to build up reference groups (i.e. standard lengths of the reacted testing solutions), the activity value of the Nattokinase solution 24 can be obtained by comparing the length of the reacted testing solution 23′ for the Nattokinase solution 24 with the standard length of reacted testing solutions. Referring to FIG. 2C, the first substrate 201 or the second substrate 202 of the assaying device 2 further has calibrations 203 thereon. With the calibrations 203, the length of the reacted testing solution 23′ can be more precisely measured, and the activity value of the Nattokinase solution 24 can be obtained quickly and conveniently.

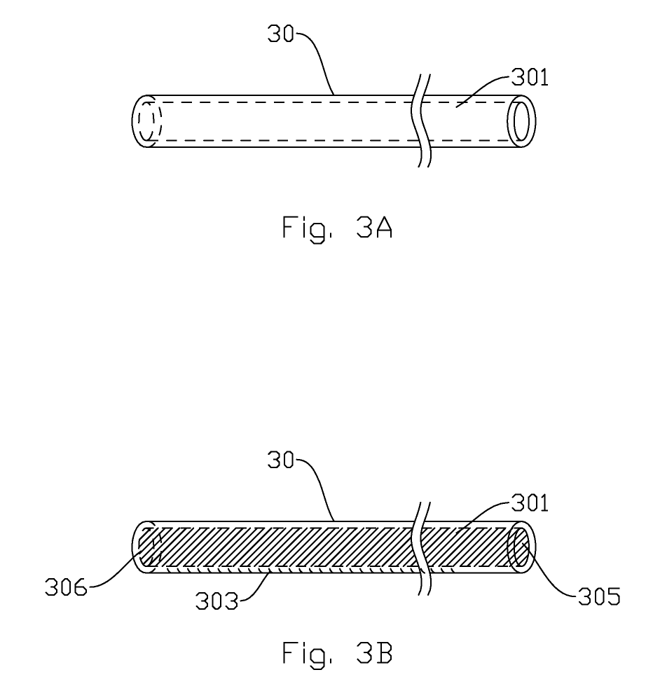

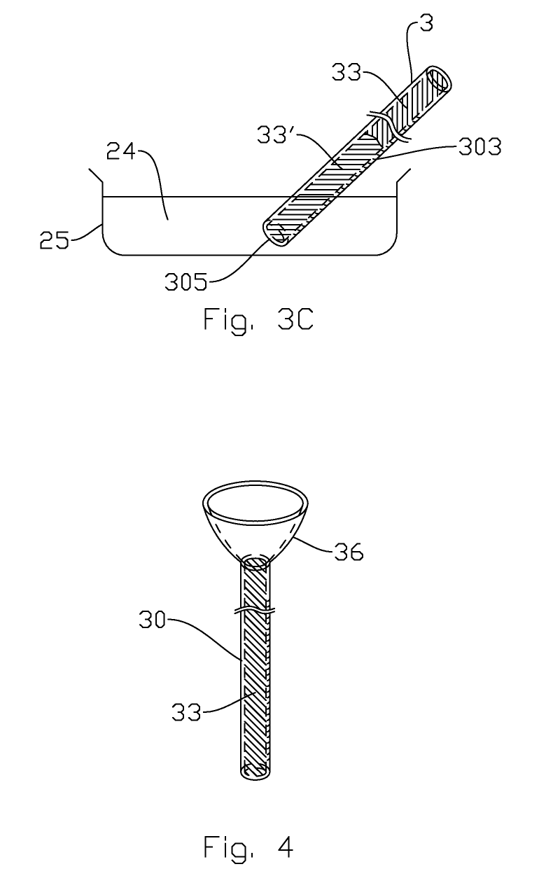

The assaying device for determining the activity value of Nattokinase solution of the second embodiment of the present invention is shown in FIGS. 3A to 3D. The assaying device 3 comprises a container 30 having a receiving room 301 and a testing solution 33 filled in the receiving room 301. The container 30 is in a long pattern and has the same cross-section in one axis extending from the one end toward the opposite end of the container. Yet it is also acceptable that the cross-section of the container 30 in the axis is not fixed if all the Nattokinase samples use the same kind of container 30 and testing solution 33. Preferably, the container 30 is a transparent and tubular container, as shown is FIG. 3A. The container 30 has a first opening 305 and a second opening 306, also referring to FIG. 3B, and the testing solution 33 is filled in the receiving room 301 from the first opening 305. The assaying device 3 is preferably used after two ends of the container 30 are cut off. Alternatively, the container 30 can only have a first opening 305 (not shown in figures) and the testing solution 33 is filled into the receiving room 301 from the first opening 305 by vacuum filling, that is, the air in the receiving room 301 is draw out to be vacuum first, and then the device 3 is placed in a receptacle having the testing solution 33 to make the testing solution 33 be suck in the receiving room 301 due to the effect of pressure difference. The testing solution 33 is the same as that in first embodiment; therefore the detail thereof is omitted.

FIG. 3C is schematic view showing the assaying device 3 applied to a Nattokinase solution 24 with unknown activity value which is placed in a tank 25. The first opening 305 of the device 3 is directly inserted in the Nattokinase solution 24. When the testing solution 33 in the first opening 305 contacts and reacts with the Nattokinase solution 24, a portion of the testing solution 33 becomes a reacted testing solution 33′ extending from the first opening 305 toward the inside of the assaying device 3. Since the reacted testing solution 33′ and the testing solution 23 have different colors, and the container 30 is transparent, one can easily observe and measure the length of the reacted testing solution 33′ through container 30. If some Nattokinase solutions with standard activity values are also taken to run the process to build up reference groups (i.e. standard lengths of the reacted testing solutions), the activity value of the Nattokinase solution 24 can be obtained by comparing the length of the reacted testing solution 23′ for the Nattokinase solution 24 with the standard length of reacted testing solutions. The container 30 also can further have calibrations 303 thereon. With the calibrations 303, the length of the reacted testing solution 33′ can be more precisely measured, and the activity value of the Nattokinase solution 24 can be obtained quickly and conveniently.

With reference to FIG. 4, the container 30 can further have an expanding entrance 36 connected with the first opening 305. When the activity value of the Nattokinase solution 24 is expected to be measured, the Nattokinase solution 24 can be directly dropped in the expanding entrance 36 and through the first opening 305 to react with the testing solution 24. The design concept of the expanding entrance 36 also can be applied to the assaying device 2 of the first embodiment.

From the first and second embodiments above, the assaying device for determining the activity value of a Nattokinase solution of present invention comprises a container which has at least one opening and has a testing solution filled therein. When the opening of the device contacts a Nattokinase solution, the testing solution will react with the Nattokinase solution and change its color. By measuring the length of the reacted testing solution, one quickly obtains the activity value of the Nattokinase solution. Furthermore, by strictly defining the receiving room of the container, the accuracy of the activity value is enhanced. In more detailed, if the container is designed in a long pattern, one can more conveniently obtain the activity value by length of the reacted testing solution.

The above description is given by way of example, and not limitation. Given the above disclosure, one skilled in the art could devise variations that are within the scope and spirit of the invention disclosed herein, including configurations ways of the recessed portions and materials and/or designs of the attaching structures. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. An assaying device for determining an activity value of Nattokinase solution, comprising:
a container comprising a first opening and a receiving room; and
a testing solution filled in the receiving room, wherein when the first opening having the testing solution contacts a Nattokinase solution, a reaction occurs in the container.

2. The assaying device for determining the activity value of Nattokinase solution of claim 1, wherein the container is in a long pattern.

3. The assaying device for determining the activity value of Nattokinase solution of claim 2, wherein the container is in a tubular shape.

4. The assaying device for determining the activity value of Nattokinase solution of claim 2, wherein the container has the same cross-section in one axis extending from the first opening toward the opposite end of the container.

5. The assaying device for determining the activity value of Nattokinase solution of claim 1, wherein the container has calibrations thereon.

6. The assaying device for determining the activity value of Nattokinase solution of claim 1, wherein the container further comprises:
a first substrate;
a second substrate disposed in parallel with the first substrate to form the receiving room therebetween; and
a plurality of spacers disposed between the first substrate and the second substrate.

7. The assaying device for determining the activity value of Nattokinase solution of claim 6, wherein the scale of spacer is not larger than 2 mm.

8. The assaying device for determining the activity value of Nattokinase solution of claim 6, wherein each of the spacers contacts the first substrate and the second substrate.

9. The assaying device for determining the activity value of Nattokinase solution of claim 6, wherein at least one of the first substrate and the second substrate is transparent.

10. The assaying device for determining the activity value of Nattokinase solution of claim 9, wherein at least one of the first substrate and the second substrate is made of glass.

11. The assaying device for determining the activity value of Nattokinase solution of claim 6, wherein there is an edge adhesive disposed between the first substrate and the second substrate.

12. The assaying device for determining the activity value of Nattokinase solution of claim 6, wherein at least one of the first substrate and the second substrate has calibrations thereon.

13. The assaying device for determining the activity value of Nattokinase solution of claim 1, wherein the container further comprises an expanding entrance connected with the first opening.

14. The assaying device for determining the activity value of Nattokinase solution of claim 1, wherein the color of the reacted testing solution is different from that of the original testing solution.

15. The assaying device for determining the activity value of Nattokinase solution of claim 1, wherein the testing solution is filled into the receiving room by means of vacuum filling.

16. The assaying device for determining the activity value of Nattokinase solution of claim 1, wherein the container further comprises a second opening located opposite the first opening, and the testing solution is filled into the receiving room from the first opening.

17. The assaying device for determining the activity value of Nattokinase solution of claim 1, wherein two ends of the container is cut off.
